(19) United States
(12) Patent Application Publication
(10) Pub. No.: US 2023/0242799 A1
(43) Pub. Date: Aug. 3, 2023

(54) Antimicrobial Moisture Avoidance Powers and Substances

(71) Applicant: ATACAMA, LLC, Lafayette, CO (US)

(72) Inventor: Jack Tuo DONG, Denver, CO (US)

(73) Assignee: ATACAMA, LLC, Lafayette, CO (US)

(21) Appl. No.: 18/011,972

(22) PCT Filed: Jun. 21, 2021
(86) PCT No.: PCT/US2021/070740

§ 371 Date: Dec. 21, 2022

Related U.S. Application Data
(60) Provisional application No. 63/042,147, filed on Jun. 22, 2020.

(51) Int. Cl. C09K 3/14; B22F 1/054; B22F 1/12; B22F 1/10

(52) U.S. Cl. CPC C09K 3/149; B22F 1/10; B22F 1/12; B22F 1/054; B22F 2301/10; B22F 2302/25; B22F 2302/45; B22F 2304/054

(57) ABSTRACT

Described herein is a composition for antimicrobial moisture avoidance powders and substances. Copper and copper alloys have been known to possess antimicrobial activity. Calcium carbonate and magnesium carbonate that is infused with chalk are effective at not only drying the skin and avoiding moisture, but also possess strong antimicrobial activity. According to one embodi-ment, a composition for a moisture avoidance powder comprises copper oxide with one or more of calcium carbonate and magnesium carbonate. The copper oxide may be included in the composition at a minimum ratio of 1 part copper oxide to 1000 parts of the combination mixture of one or more of calcium carbonate and magnesium carbonate. That composition can be combined with alcohol to produce a moisture avoidance substance in a liquid or cream form with antimicrobial properties.

### PRIORITY CLAIM

The present invention is related to, and claims priority from, U.S. Provisional Pat. Application Ser. No. 63/042,147, filed on Jun. 22, 2020, the disclosure of which is hereby incorporated by this reference in its entirety.

### FIELD OF THE INVENTION

The present application is directed to antimicrobial moisture avoidance powders and substances.

### BACKGROUND OF THE INVENTION

Moisture avoidance powders and substances are regularly used and necessary in such activities as exercising, powerlifting, weightlifting, bodybuilding, gymnastics, rock climbing, pole vaulting, and pole dancing. In particular, either calcium carbonate (CaCO₃) and magnesium carbonate (MgCO₃), more commonly known as chalk, or a combination thereof, is commonly used in such activities to dry the hands and create friction between the skin and the surface of the desired item to be put in contact with. For example, chalk assists a powerlifter to better hold on to a bar during deadlifts or pullups. Similarly, chalk assists a rock climber with grasping the rock when climbing outside or grasping artificial holds when climbing in an indoor gym.

Such “weightlifting,” “climbing,” or “exercise” chalk can be commonly found in various forms. One of the more popular forms is as “loose” chalk that has been ground or filtered to be very fine powder form. Chalk also comes in a “block” form - a solid, compressed form. A block of chalk is more similar to the chalk one would use on a chalkboard. Chalk can also be found packed into chalk “balls” or “socks.” The ball or sock is made of a thin material and releases chalk incrementally through the material when the ball or sock is squeezed. Chalk also comes in a “liquid” form. The chalk is usually mixed with alcohol, water and other agents (such as resin or a thickening agent). When the liquid chalk is applied to hands, the alcohol evaporates quickly and leaves a dried, evenly chalked hand. When the alcohol is at a higher concentration in the mix, the alcohol also acts as a disinfectant. Chalk can also be found in a “cream” form that is basically a thicker and faster drying version of liquid chalk. Cream chalk generally is a mixture between calcium carbonate, magnesium carbonate with water and alcohol. Again, when the alcohol and water evaporates, all that is left on the hands is pure chalk. Similar to liquid chalk, cream chalk disinfects the hands before use when the alcohol is at a higher concentration in the mix.

Although chalk is effective at drying hands, particularly for exercise and other fitness uses, chalk does not itself possess any known antimicrobial (including antibacterial and antiviral) properties. Chalk, when combined with a high levels of alcohol, is able to briefly disinfect the hand. However, the alcohol chalk combination does not continue to have any antimicrobial activity once the alcohol evaporates. Nor does the alcohol chalk combination, which is solely used on the hand, assist with any antimicrobial activities on the desired surfaces that the hands will make contact with. In other words, the alcohol chalk provides no benefit, for example, the commonly communally used weights or holds in a climbing gym.

In order for individuals to continue to participate and safely enjoy sports and exercise, an improved solution for moisture avoidance powder and/or substance possessing antimicrobial properties is desired, particularly given the prevalence of easily transmittable human-to-human bacteria and viruses, such as influenza “flu” viruses, severe acute respiratory syndrome coronavirus (SARS-CoV or SARS-CoV-1), MERS-coronavirus (MERS-CoV), and the severe acute respiratory syndrome coronavirus 2 (SARS-CoV-2). In particular, a solution that both protects the individual using the moisture avoidance powder and/or substance and protects the contact surface for the enjoyment of use by others is desired.

### SUMMARY OF THE INVENTION

Embodiments described herein are directed to compositions of antimicrobial moisture avoidance powders and substances. Copper and copper alloys have been known to possess antimicrobial activity. Calcium carbonate and magnesium carbonate that is infused with copper oxide are effective at drying the skin and avoiding moisture and also possess strong antimicrobial activity. Nanoparticle residues left on the surface of contact by the copper chalk users will also provide sustained antimicrobial activities on the surface of contact for further use by other users.

According to one embodiment, a composition for a moisture avoidance powder comprises copper oxide with one or more of calcium carbonate and magnesium carbonate. The copper oxide may be included in the composition at a minimum ratio of 1 part copper oxide to 1000 parts of the combination mixture of one or more of calcium carbonate and magnesium carbonate.

According to another embodiment, a composition for a moisture avoidance substance comprises alcohol, copper oxide with one or more of calcium carbonate and magnesium carbonate. The copper oxide may be included in the composition at a minimum ratio of 1 part copper oxide to 1000 parts of the combination mixture of one or more of calcium carbonate and magnesium carbonate. The alcohol is mixed with the copper oxide carbonate mixture at a ratio of a minimum of 30% in volume of alcohol to copper oxide carbonate mixture.

According to another embodiment, a composition for an antimicrobial moisture avoidance substance comprises alcohol, copper oxide, one or more of calcium carbonate and magnesium carbonate, and one or more of ascorbic acid, hydrogen peroxide, and cysteine. The copper oxide may be included in the composition at a minimum ratio of 1 part copper oxide to 1000 parts of the combination mixture of one or more of calcium carbonate and magnesium carbonate. The alcohol is mixed with the copper oxide carbonate mixture at a ratio of a minimum of 30% in volume of alcohol to copper oxide carbonate mixture.

The moisture avoidance powder and substance compositions described herein can be incorporated into various forms of “chalk” without limitation, including but not limited to being loose and powdered, compressed into a block, stored in bags, as liquids or as creams.

### BRIEF DESCRIPTION OF THE DRAWINGS

FIG. 1 is a diagram showing the rapid inactivation of human coronaviruses on copper surfaces over time.

FIG. 2 is a diagram showing the effective inactivation of bacteria at different concentrations of copper in copper-based nanoparticle chalk.

### DETAILED DESCRIPTION OF THE INVENTION

The use of numerical values in the various ranges specified herein, unless otherwise expressly indicated otherwise, are considered to be approximations as though the minimum and maximum values within the stated ranges were both preceded by the word “about.” In this manner, it should be understood that slight variations above and below the stated ranges can be used to achieve substantially the same results as the values within the ranges. Additionally, the disclosure of these ranges is intended as a continuous range including every value between the minimum and maximum values.

An effective solution for items with antimicrobial properties is needed, particularly given the prevalence of easily transmittable human-to-human bacteria and viruses, such as influenza “flu” viruses, severe acute respiratory syndrome coronavirus (SARS-CoV or SARS-CoV-1)—which causes severe acute respiratory syndrome (SARS), MERS-coronavirus (MERS-CoV)—which causes Middle East Respiratory syndrome (MERS), and the severe acute respiratory syndrome coronavirus 2 (SARS-CoV-2)—which causes coronavirus disease 2019 (COVID-19). In a prior study, researchers found that human coronaviruses, such as those responsible for SARS and MERS, persist in an infectious state on common surface materials, for several days. See Warnes SL, Little ZR, Keevil CW. Human Coronavirus 229E Remains Infectious on Common Touch Surface Materials. mBio. 2015;6(6):e01697-15. Published 2015 Nov 10. doi:10.1128/mBio.01697-15 (“Warnes”). Human coronaviruses, for example, have been found to last at least 5 days on glass, stainless steel, ceramic tiles, PVC, and other materials.

Copper is a naturally occurring element that has never been found to cause cancer or any adverse effects on humans. Copper and copper alloys are also known to possess significant antimicrobial activity. Copper has been found to be effective at inactivating viruses and bacteria, such as human coronaviruses, herpes simplex virus (HSV), MRSA (Methicillin-Resistant Staphylococcus aureus), Influenza A, Norovirus, Staphylococcus aureus, Enterobacter aerogenes, Pseudomonas aeruginosa, Escherichia coli O157:H7 (E. Coli), VRE (vancomycin resistant enterococci). A study in 2020 found that found that SARS-CoV-2, the virus responsible for the coronavirus pandemic, is no longer infectious on copper within four hours, whereas SARS-CoV-2 can survive on plastic surfaces for 72 hours.

It has been proven that copper alloys, possessing at least 70% in copper, are very effective at inactivating human coronavirus. See Warnes. Copper alloys with higher concentration of coppers are known to have stronger antimicrobial activity. That is, when the concentration of copper was higher, the inactivation of the human coronaviruses was far faster.

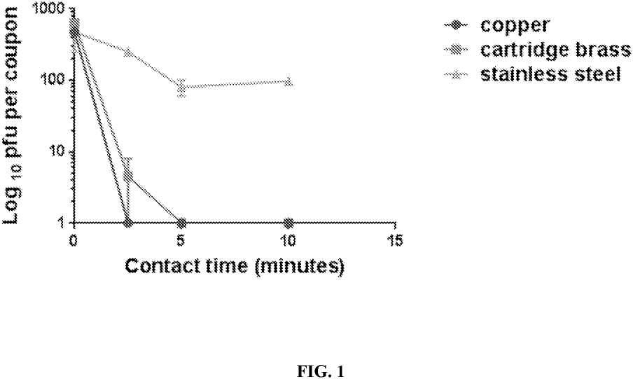

The rate of inactivation is at least eight times faster when the contamination is slight, such as a mere fingertip touch. When the amount of human coronavirus was slight, copper was found to completely inactivate human coronaviruses in just a couple of minutes. FIG. 1 reflects the very rapid inactivation of human coronaviruses on copper surfaces when the amount of human coronaviruses simulated a fingertip touch contamination.

In addition to the inactivation of microbial activity, copper oxide particles have been found to improve the well-being of the skin. Studies have shown that copper oxide particles, when embedded in socks, are able to cure athlete’s foot infections and improve skin elasticity, which is especially important for individuals suffering from diabetes. Copper oxide particles are also effective at reducing facial fine line and wrinkles when the particles are embedded in pillowcases. Moreover, copper oxide particles have been found to enhance wound healing when embedded in wound dressings.

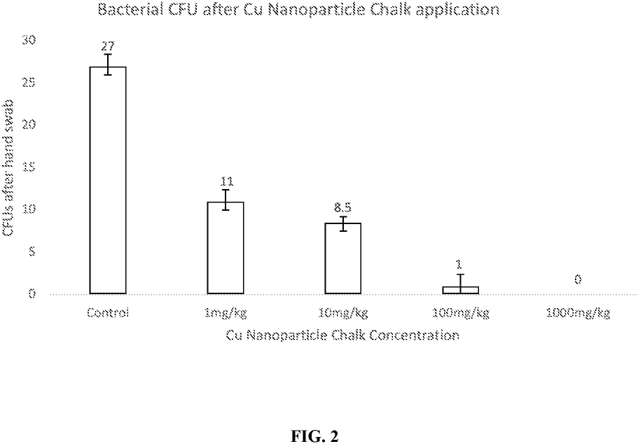

When copper oxide is mixed with calcium carbonate and/or magnesium carbonate and that mixture is brushed on hands, a mixture of a minimum ratio of 1 part copper oxide to 1000 parts of the combination mixture of one or more of calcium carbonate and magnesium carbonate (or approximately 0.1% copper oxide and 99.9% calcium carbonate and/or magnesium carbonate) was found to effectively inactivate microbial activity. FIG. 2 reflects the inactivation of microbial activity at different concentrations of copper in copper-based nanoparticle chalk. As reflected by FIG. 2, copper-based nanoparticle chalk with a copper concentration of at least 0.1% that was brushed on the hands is fairly effective at inactivating microbial activity. Copper-based nanoparticle chalk with a copper concentration of at least 0.01% that was brushed on the hands is completely effective at inactivating microbial activity.

According to one embodiment, a composition for an antimicrobial moisture avoidance powder comprises copper oxide with one or more of calcium carbonate and magnesium carbonate. The copper oxide may be included in the composition at a minimum ratio of 1 part copper oxide to 1000 parts of the combination mixture of one or more of calcium carbonate and magnesium carbonate (or approximately a minimum concentration of 0.1% copper oxide). It is a preferred embodiment to have a minimum concentration of at least 1 part copper oxide to 10000 parts of the combination mixture of one or more of calcium carbonate and magnesium carbonate (or approximately a minimum concentration of 0.01% copper oxide) to ensure complete inactivation of microbial activity. Such a copper oxide nanoparticle chalk composition, which will be referred to as “Copper Chalk” hereinafter, is effective at drying the skin, improving the skin, avoiding moisture, and possessing strong antimicrobial activity. For example, the copper oxide used in the instant invention could be any one or more of the following: copper(I) oxide (also known as cuprous oxide with the formula Cu₂O, copper(II) oxide (also known as cupric oxide with the formula CuO), copper peroxide (with the formula CuO₂), copper(III) oxide (with the formula CU₂O₃), and copper(IV) oxide (with the formula CuO₂).

In order to improve the effectiveness of the copper oxide particles, the copper oxide particles should ideally be small in size to provide additional surface area to volume contact to microbes. The copper oxide particles should be no larger than 40 nm. However, copper oxide particles that are too small have been shown in trials on animals to potentially cause irritations to the lung. Thus, the copper oxide particles should be between 20 to 40 nm in size to provide effective antimicrobial activity while remaining safe in case of human inhalation.

Similarly, chalk particles that are too small could cause irritations to the lung if inhaled in large quantities by humans. Thus, the chalk particles should also be a minimum of 20 nm in size to remain safe in case of human inhalation.

Copper Chalk could be incorporated into various other products and various other forms of “chalk.” For example, such a composition could be produced in a “loose chalk” manner. Such a composition could also be tightly packed and compressed into a “block” form. Similarly, the chalk could be packaged within “chalk bags,” bags that are made from thin slightly permeable material that lets loose chalk out when pressure is applied to the bag.

Copper Chalk could also be incorporated into a liquid or cream form. Liquid chalk is generally made by incorporating alcohol with powdered chalk. According to another embodiment, a composition for an antimicrobial moisture avoidance substance comprises alcohol, copper oxide, and one or more of calcium carbonate and magnesium carbonate. The copper oxide may be included in the composition at a minimum ratio of 1 part copper oxide to 1000 parts of the combination mixture of one or more of calcium carbonate and magnesium carbonate (or approximately 0.1% copper oxide and 99.9% calcium carbonate and/or magnesium carbonate). The alcohol is mixed with the copper oxide carbonate mixture at a ratio of a minimum of 30% in volume alcohol to copper oxide carbonate mixture to achieve a proper “liquid” consistency.

A study has found that the effectiveness of copper in inactivating microbes is further enhanced when the copper was added to the following aqueous reducing agents: ascorbic acid, hydrogen peroxide, and cysteine. According to another embodiment, a composition for an antimicrobial moisture avoidance substance comprises alcohol, copper oxide, one or more of calcium carbonate and magnesium carbonate, and one or more of ascorbic acid, hydrogen peroxide, and cysteine. The copper oxide may be included in the composition at a minimum ratio of 1 part copper oxide to 1000 parts of the combination mixture of one or more of calcium carbonate and magnesium carbonate (or approximately 0.1% copper oxide and 99.9% calcium carbonate and/or magnesium carbonate). The alcohol is mixed with the copper oxide carbonate mixture at a ratio of a minimum of 30% in volume alcohol to copper oxide carbonate mixture to achieve a proper “liquid” consistency.

Ethyl alcohol (or ethanol), at concentrations of 60%-80%, is a potent antiviral agent. Similarly, isopropyl alcohol (or isopropanol) is considered an effective antiviral agent at concentrations of 70% or more. Thus, a higher concentration of alcohol could be added to Copper Chalk for additional antimicrobial capabilities. A desirable modification would be to use alcohol that is a minimum of 60% ethanol or 70% isopropanol when adding the alcohol to the copper oxide carbonate mixture.

The various embodiments described herein are effective at drying the skin and avoiding moisture while also possessing rapid and strong antimicrobial activity on human skin. Additionally, the copper-based nanoparticle residues left on the surface of contact by the copper chalk users will contain to provide active sustained antimicrobial activities on the surface of contact, ensuring that any microbes left on the surface of contact are inactivated before further use by other users.

1. A composition for moisture avoidance, comprising:
particles of copper or a copper compound; and
a drying agent.

2. The composition of claim 1, wherein the particles of copper or a copper compound comprise between 0.001% and 0.1% by weight of the moisture avoidance composition.

3. The composition of claim 2, wherein the particles of copper or a copper compound comprise about 0.01% by weight of the moisture avoidance substance.

4. The composition of claim 1, wherein the particles of copper or a copper compound comprise at least 0.01% by weight of the moisture avoidance substance.

5. The composition of claim 1, wherein the particles of copper or a copper compound comprise copper oxide.

6. The composition of claim 1, wherein the drying agent comprises one or more of calcium carbonate and magnesium carbonate.

7. The composition of claim 1, wherein the particles of copper or a copper compound are at least 20 nm in diameter.

8. The composition of claim 7, wherein the particles of copper or a copper compound are at most 40 nm in diameter.

9. The composition of claim 1, further comprising alcohol.

10. The composition of claim 9, wherein the alcohol comprises a minimum of 30% of the composition by volume.

11. The composition of claim 10, wherein the alcohol comprises ethanol or isoporopanol.

12. The composition of claim 11, wherein the alcohol comprises a minimum of 60% ethanol by weight.

13. The composition of claim 11, wherein the alcohol comprises a minimum of 70% isopropoanol by weight.

14. The composition of claim 9, further comprising an aqueous reducing agent.

15. The composition of claim 14, wherein the aqueous reducing agent comprises one or more of ascorbic acid, hydrogen peroxide, and cysteine.

16. The composition of claim 9, wherein the particles of copper or a copper compound are present in a ratio of 1 part copper or copper compound to 1000 parts of the drying agent.

17. The composition of claim 1, wherein the composition is in the form of any one of a loose powder, a compressed block, a liquid and a cream.

18. The composition of claim 9, wherein the composition is in the form of any one of a liquid and a cream.

19. A permeable bag or sock containing the composition of claim 17 in the form of a loose powder.

20. A method for producing a moisture avoidance substance comprising a step of:
mixing particles of copper or a copper compound with a drying agent.
